(19) United States
(12) Patent Application Publication
(10) Pub. No.: US 2020/0289105 A1
(43) Pub. Date: Sep. 17, 2020

(54) DILATOR SYSTEM AND A METHOD OF USING A DILATOR SYSTEM

(71) Applicant: Mohammad ETMINAN, Houston, TX (US)

(72) Inventor: Mohammad ETMINAN, Houston, TX (US)

(21) Appl. No.: 16/818,023

(22) Filed: Mar. 13, 2020

Related U.S. Application Data
(60) Provisional application No. 62/817,721, filed on Mar. 13, 2019.

(51) Int. Cl. A61B 17/02; A61M 29/00

(52) U.S. Cl. CPC A61B 17/025; A61B 2017/0046; A61B 2017/0256; A61M 29/00

(57) ABSTRACT

Disclosed is a dilator system that includes a first tube having an eccentrically located guidewire opening radially spaced from a central longitudinal axis of the first tube, and having a first length, and a second tube being nestable inside the first tube and having a second length longer than the first length. Also disclosed is a method of dilating tissue with the system that includes guiding the first tube toward a surgical area of interest while dilating the tissue.

### CROSS-REFERENCE TO RELATED APPLICATIONS

The present application claims priority to U.S. Provisional Application Ser. No. 62/817,721, filed Mar. 13, 2019, the entire disclosure of which is incorporated by reference herein.

### FIELD OF THE INVENTION

The disclosed invention relates to a dilator system and a method of using a dilator system.

### BACKGROUND

A disc herniation may be removed through discectomy to relieve pain by removing pressure on a nerve.

A traditional method of discectomy requires an up to four inches long incision down the back and stripping of muscle from the spine to access the herniated disc. Microsurgery may be implemented with a one inch incision, but still requires cutting muscle and scraping it from the spine. The muscle damage causes post-operative pain and requires longer rehabilitation periods.

A surgical tool having metal tubes of various inner diameters has been proposed to create and maintain openings to access the spine. The metal tubes are called dilators, which progressively increase in diameter size. The dilators are inserted sequentially to gradually separate the muscle to create an opening large enough for surgical tools. Thus, a surgeon may avoid cutting the muscle fiber. The METRx system is an example of such a system. The use of such systems is discussed in Clinical Applications of the Tubular Refractor on Spinal Disorders, Kim et al., J Korean Neurosurgery Soc. 2007, October, 42(4) 245-250.

FIG. 1 shows the dilators 10 of a system 12 according to the prior art. This system is called METRx. As seen, each dilator 10 is a tube with two opposite ends and has a diameter different from the other dilators 10 such that one dilator 10 can be received inside of another dilator 10. The sequential insertion of the dilators permits the gradual dilating of the muscle.

According to conventional art, a dilator tube 10 can be guided to the surgery site with a guide wire. The guide wire is secured to a spinal site using any known technique, for example, a known technique for the insertion of percutaneous screws. According to this known technique a dilator tube receives the secured guide wire in the interior space thereof and is guided to the surgery site.

### SUMMARY OF THE INVENTION

An objective of a system according to the present invention is to facilitate performance of the posterior lumbar fusion based on a guidewire secured in order to insert percutaneous screws.

A system according to the present invention will use the secured guidewire as a reference point for the insertion of the dilators in order to appropriately locate the facet joint of interest in the lumbar and thoracic spine to allow for the percutaneous, automated preparation of the facet joint and the delivery of grafting material on various sources including but not limited to autograft, allograft, demineralized bone matrix, various ceramics, biologics, or variations thereof.

A system according to the present invention includes a novel dilation mechanism which will allow the tightening of the dilator based on an eccentrically positioned guidewire pathway on the outer periphery of a dilator, which will preferably serve as the surgical cannula. This is contrary to the conventional method in which the guidewire is centered in the middle of the dilator. The eccentric guidewire pathway allows for a lateral/medial/superior/inferior placement of the dilator (cannula) and access to the points of interest medial/lateral/superior/inferior to the guidewire entry site.

To perform a surgical method with a system according to the present invention, the guidewire may be secured through a conventional method, which could include the use of a needle to allow cannulation of the interested anatomy such as the pedicle in the case of the lumbar, and thoracic spine.

The guidewire, which may be a flexible body, can be placed for the purposes of the preparation and the insertion of the dilator (cannula) in question or for the placement of pedicle screws by the use of fluoroscopy, navigation of various sorts, direct visualization, and the like.

Once the guidewire is placed (for example, through the use of possible insertion needles), the guidewire is received in the eccentric guidewire pathway of the dilator (cannula), and the dilator (cannula) is guided to the desired site by sliding the eccentric guidewire pathway along the guidewire.

A dilator system according to the present invention includes a first tube having an eccentrically located guidewire opening radially spaced from a central longitudinal axis of the first tube, and having a first length, and a second tube being nestable inside the first tube and having a second length longer than the first length.

In the first embodiment, the first tube may have a first interior diameter, and a first exterior diameter, and the second tube may have a second interior diameter, and a second exterior diameter smaller than the first interior diameter. In the first embodiment, the first inner diameter may be dimensioned so that the first tube may serve as a cannula.

In a second embodiment, the first tube may be radially expandable to a first diameter to permit the nesting of the second tube therein. Thus, the first tube may include a plurality of pieces connected with a spring to become radially expandable. The plurality of pieces may be two semi-cylindrical pieces. In this embodiment, the second tube has a second inner diameter dimensioned so that the second tube may serve as a cannula.

A system according to the present invention may further include a third tube having a third interior diameter, a third exterior diameter smaller than the second interior diameter and a third length longer than the second length.

A lobe may be on an exterior surface of the first tube defining the guidewire opening.

A plurality of teeth may be located and extend from a distal end of the tube serving as the cannula.

A handle may be detachably attachable to the tube serving as the cannula.

A method according to the present invention includes dilating tissue with a dilator system according to the present invention by attaching a guide wire at a surgical area of interest within the human body, receiving the guidewire in the guidewire opening, and dilating the tissue by advancing the first tube along the guidewire toward the surgical area of interest.

The method may include nesting the second tube within the first tube prior to advancing the first tube toward the surgical area of interest, and dilating the tissue with the second tube prior to dilating the tissue with the first tube by advancing the second tube toward the surgical area of interest.

The first tube may be radially expandable to have a variable diameter, and the method may further include receiving the second tube inside the first tube and radially expanding the first tube prior to advancing the first tube toward the surgical area of interest, and dilating tissue by advancing the second tube toward the surgical area of interest prior to advancing the first tube.

The surgical area of interest may be the site of a facet joint, and the first tube or the second tube may be dimensioned to serve as a cannula, which is used for preparing the facet joint through the cannula.

The method may further include docking the cannula at the surgical area of interest and stabilizing the cannula with an arm or a handle prior to preparing the facet joint.

The method may further include dissecting the tissue with the first tube while advancing the first tube toward the surgical area of interest.

Other features and advantages of the present invention will become apparent from the following description of the invention which refers to the accompanying drawings.

### BRIEF DESCRIPTION OF THE DRAWINGS

FIG. 1 shows a system according to the prior art.

FIG. 2 shows a dilator system according to the present invention assembled on site, prior to removal of the inside dilators.

FIG. 3 shows a dilator serving as a cannula in a system according to the present invention.

FIG. 4 shows a top plan view of a cannula according to the present invention.

FIG. 5 shows a side view of a dilator serving as the cannula with a handle.

FIG. 6 shows a side view of a variable diameter dilator.

FIG. 7 shows a top plan view of the variable diameter dilator of FIG. 6.

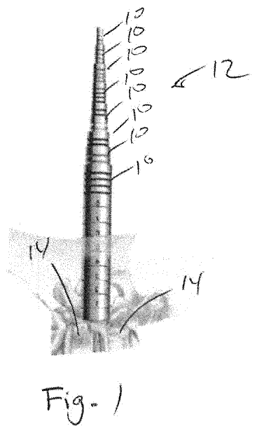

### DETAILED DESCRIPTION OF THE INVENTION

FIG. 2 shows a system according to the present invention in a deployed state prior to removal of the inside dilators.

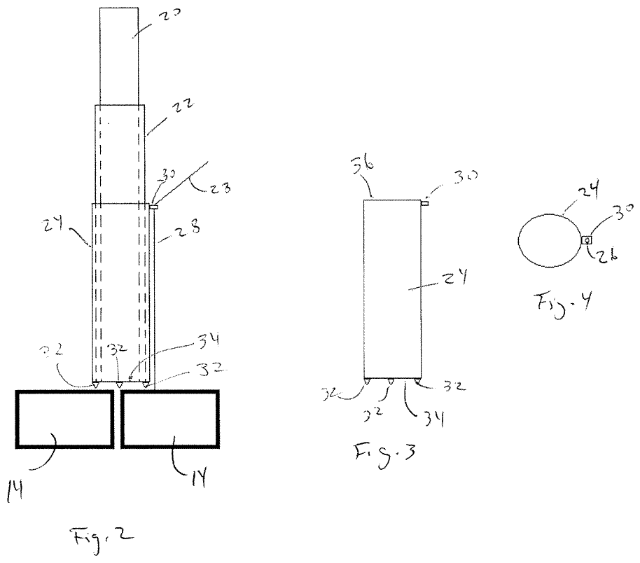

Referring to FIGS. 2, 3 and 4, a system according to the present invention includes a plurality of dilators, for example, three dilators 20, 22, 24. Each dilator 20, 22, 24 is preferably a tube with an interior diameter and an exterior diameter. The interior diameter and the exterior diameter of each dilator are different than the interior diameter and the exterior diameter of the other dilators. The dilator 24 with the largest exterior and interior diameters serves as the cannula for performing the operation, and, for convenience, will be referred to as the cannula hereafter.

According to the present invention, the cannula 24 will have a guide hole (pathway) 26 sized to receive a guide wire 28 that is secured to a location on a vertebrae 14. The guide wire hole 26 may be defined in, for example, a lobe 30 extending radially away from the exterior surface of the cannula 24. Thus, the guide hole 26 is at an eccentric location outside of the interior space of the cannula 24.

The cannula 24 may also have a plurality of circumferentially spaced teeth 32 at its distal end 34 to engage the area on the vertebra around the site of operation.

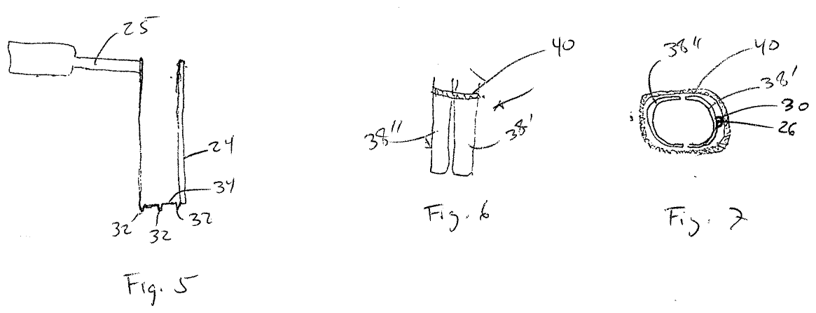

The cannula 24 may also include a handle 25 (FIG. 5), which may be detachably attached to the cannula 24 at or near its proximal end 36.

As seen in FIG. 2, the cannula 24 is the shortest dilator in the system. In the system shown in FIG. 2, the longest dilator 20 has the narrowest internal and external diameters, and the dilator 22 that is longer than the cannula 24 but shorter than the longest dilator 20 has an external diameter that is smaller than the internal diameter of the cannula 24, and an internal diameter that is wider than the exterior diameter of the longest dilator 20. Thus, dilator 22 can nest in dilator (cannula) 24 and dilator 20 can nest in dilator 22.

The first embodiment may include two or three dilator tubes 20, 22, 24, in which the dilator tube 24 with the largest diameter will have the eccentric guidewire pathway 26. The other one or two dilator tubes 20, 22 have smaller diameters and are receivable in the dilator tube 24 with the largest diameter and the eccentric guidewire pathway 26. The one or two internal dilators 20, 22 allow for dilatation of the muscle in sequence. Specifically, the narrowest tube 20 is inserted first. Then, the second tube 22 with the intermediate height and diameters is received over the narrowest tube 20. Thereafter, the widest tube 24 receives the guide wire 28 in pathway 26 and guided to the surgery site over the other two, already inserted, dilators 20, 22.

In the first embodiment, dilator 20 is inserted into dilator 22, and then the two dilators 20, 22 are inserted into dilator 24. In this assembled (nested) state, the guidewire is received in guidewire pathway 26 of dilator 24, and the assembly of the dilators 20, 22, 24 is guided, during the initial phase of the insertion, past skin, subcutaneous fat, and fascia. If necessary, with dilator 24 and its guide mechanism acting as a guide of general direction, dilators 20 and 22 will be advanced on to the area of special interest (the surgery area) in order to allow for a final muscle dissection. Once dilators 20, 22 have been advanced and docked, dilator 24 will be advanced over them and docked around the area of interest. Dilators 20 and 22 will be then removed. Once the muscle is properly dilated, and the internal dilators 20, 22 are removed, the largest diameter dilator 24 will now work as a working cannula. The internal dilators 20, 22 are longer than the cannula 24 so that once the initial fascia and musculature is centered the longer two dilators 20, 22 can be advanced to the facet joint or the area of interest and docked. The final working dilator 24 has teeth/structures 32 to allow for the engagement of the anatomy of interest and appropriate docking.

Referring to FIGS. 6 and 7, in the second embodiment, an expandable dilator 38 with a variable diameter is used. The expandable dilator 38 includes at least two preferably semi-cylindrical pieces 38′, 38″ held together by a spring mechanism 40 disposed around the pieces 38′, 38″. The spring mechanism 40 allows for the radial expansion of the pieces 38′, 38″ upon insertion of additional dilators therein. The expandable dilator 38 also includes a lobe 30 with a guidewire pathway (hole) 26 on an outer surface of one of the pieces 38′, 38″ at or near the proximal end to allow for guidance using the guidewire 28. The expandable dilator 38 is advanced to the anatomy of interest (which would be medial/lateral/superior/inferior to the secured guidewire 28) with a loud talking and possible aggressive dissection of the soft tissue structures anterior to its docking teeth/edge. Once the variable diameter dilator 38 is docked, additional dilators 20, 22, 24 would be inserted therein to dissect the soft tissue gradually. In the second embodiment, once the guidewire 28 is inserted through the pathway 26, the expandable dilator 38 is guided to the location of interest and docked. Expandable dilator 38 is expanded using gradually larger diameter dilators (for example, dilators 20, 22, 24) up to the size of the final working cannula 24. The final working cannula 24 will remain inside the dilator 38 as the smaller dilators 20, 22 are removed. The final working assembly would include the expandable dilator 38 and the largest diameter dilator 24 that was inserted as the final piece of dilation. The final construct would be held and located using the initial inserted guidewire 28. The distal tip of the expandable dilator 38 may be designed to allow an aggressive dissection of the soft tissue as it is expanded outwardly, or designed not to aggressively dissect the soft tissues. The final dilator (cannula 24) inserted within the variable diameter dilator 38 would be the working cannula. The proximal/posterior edge of the expandable dilator 38 is designed to accept the placement of additional dilators.

In both embodiments, the final working dilator 24, which serves as the working cannula 24, would have an attached (which maybe detachable) handle (FIG. 5) to allow stabilization externally or would have a possible connection mechanism to an arm anchored to a stable point.

Once docked onto the structure the soft tissue is removed if necessary. The drill guide with drill stops that are variable and can be adjusted based on the surgeons preference will work through the final working cannula 24 in order to appropriately decorticate and prepare the structure of interest such as a facet joint in the thoracic and lumbar spine.

The mechanism of the drill/mill will be such that a smooth operation will be allowed within the working cannula 24.

Various parts can be used to place fusion material onto the appropriately prepared facet joint in order to allow solid fusion.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A dilator system, comprising:
a first tube having an eccentrically located guidewire opening radially spaced from a central longitudinal axis of the first tube, and having a first length; and
a second tube being nestable inside the first tube and having a second length longer than the first length.

2. The dilator system of claim 1, wherein the first tube has a first interior diameter, and a first exterior diameter, and the second tube has a second interior diameter, and a second exterior diameter smaller than the first interior diameter.

3. The dilator system of claim 2, further comprising a third tube having a third interior diameter, a third exterior diameter smaller than the second interior diameter and a third length longer than the second length.

4. The dilator system of claim 1, further comprising a lobe located on an exterior surface of the first tube having the guidewire opening defined therein

5. The dilator system of claim 1, further comprising a plurality of teeth located and extending from a distal end of the first tube.

6. The dilator system of claim 1, further comprising a handle detachably attachable to the first tube.

7. The dilator system of claim 1, wherein the first tube is radially expandable to a first diameter to permit nesting of the second tube therein.

8. The dilator system of claim 6, wherein the first tube comprises a plurality of pieces connected with a spring to become radially expandable.

9. The dilator system of claim 7, wherein the plurality of pieces are two semi-cylindrical pieces.

10. The dilator system of claim 7, wherein the second tube has a second inner diameter dimensioned so that the second tube may serve as a cannula.

11. The dilator system of claim 2, wherein the first inner diameter is dimensioned so that the first tube may serve as a cannula.

12. A method of dilating tissue with a dilator system that includes a first tube having an eccentrically located guidewire opening radially spaced from a central longitudinal axis of the first tube and a first length, and a second tube nestable inside the first tube and having a second length longer than the first length, the method comprising:
attaching a guide wire at a surgical area of interest within a human body;
receiving the guidewire in the guidewire opening; and
dilating the tissue by advancing the first tube along the guidewire toward the surgical area of interest.

13. The method of claim 11, further comprising nesting the second tube within the first tube prior to advancing the first tube toward the surgical area of interest, and dilating the tissue with the second tube prior to dilating the tissue with the first tube by advancing the second tube toward the surgical area of interest.

14. The method of claim 13, wherein the surgical area of interest is a site of a facet joint, and wherein the first tube is dimensioned to serve as a cannula, and further comprising preparing the facet joint through the cannula.

15. The method of claim 13, further comprising docking the cannula at the surgical area of interest and stabilizing the cannula with an arm or a handle prior to preparing the facet joint.

16. The method of claim 11, wherein the first tube is radially expandable to have a variable diameter, and further comprising receiving the second tube inside the first tube and radially expanding the first tube prior to advancing the first tube toward the surgical area of interest, and dilating tissue by advancing the second tube toward the surgical area of interest prior to advancing the first tube.

17. The method of claim 16, wherein the surgical area of interest is a site of a facet joint, and wherein the second tube is dimensioned to serve as a cannula, and further comprising preparing the facet joint through the cannula.

18. The method of claim 15, further comprising docking the cannula at the surgical area of interest and stabilizing the cannula with an arm or a handle prior to preparing the facet joint.

19. The method of claim 15, further comprising dissecting tissue with the first tube while advancing the first tube toward the surgical area of interest.
